(19) United States
(12) Patent Application Publication
(10) Pub. No.: US 2010/0133316 A1
(43) Pub. Date: Jun. 3, 2010

(54) INSTRUMENT AND ASSEMBLY FOR FITTING A COMPRESSION STAPLE

(76) Inventors: Emmanuel Lizee, Saint Ismier (FR); Christophe Magnac, La Rochette (FR)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(21) Appl. No.: 12/626,228

(22) Filed: Nov. 25, 2009

(30) Foreign Application Priority Data
Nov. 28, 2008 (FR) 08 58093

(51) Int. Cl. A61B 17/10

(52) U.S. Cl. 227/175.1

(57) ABSTRACT

An instrument including a distal receiver portion for receiving a compression staple that includes two branches interconnected by a transverse bridge. The instrument further including a holder suitable for holding a first zone of the bridge of a staple stationary relative to the receiver portion, the first zone being either the two end portions of the bridge that are connected to the branches or the central portion of the bridge. The instrument further including a deformation tool for deforming a staple from an initial configuration for implanting in a bone towards a compression-applying configuration, said deformation tool adapted to apply forces to the second zone of the bridge of the staple producing a resultant that opposes the forces applied to the first zone by the holder.

### CROSS REFERENCE TO RELATED APPLICATIONS

This application claims priority under 35 U.S.C. §§119(a)-(d) to French Patent Application No. 08 58093 filed on Nov. 28, 2008, the entire contents of which are incorporated herein by reference.

### FIELD

Embodiments of the present invention relate to an instrument for fitting compression staples, to an assembly for fitting a compression staple, and to a method of fitting a compression staple to compress, fuse, join, span, and/or unite two bone portions.

### BACKGROUND

A compression staple is used to hold two bone portions in a relative position in order to reinforce them and hold them in position. Such a compression staple conventionally comprises two branches interconnected by a transverse bridge, the branches being designed to be inserted in the bone portions on either side of the site of a fracture or an osteotomy in a bone that is to be repaired. The two branches of such a staple are designed to be moved towards each other so as to enable them to press the two bone portions against each other with a certain amount of pressure.

EP-A-1 870 042 describes a device for fitting compression staples that are made of a shape memory material, with branches that converge in the rest configuration of the staple. That device comprises a spreader part of trapezoidal shape for spreading the initially converging branches of the staple into a configuration for implanting the staple in a bone. The spreader part is positionable, while a staple is being fitted, between the branches of the staple on the side of the bridge that faces towards the branches. While the staple is being fitted, it is initially impacted partially into the bone portions, while the branches are held apart by the spreader part. The spreader part is then separated from the staple. Finally, the staple is impacted fully into the bone portions. Such a device thus requires two successive steps of impacting the staple, thereby lengthening the time taken to implant it. Furthermore, a staple made of shape memory material does not permit control of the compression load that is applied to the bone portions when the staple is in its implanted configuration.

### SUMMARY

Embodiments of the present invention include an instrument and an assembly for fitting compression staples that permit a compression staple to be implanted easily in bone portions for consolidation (e.g. compression and/or fusing), with the time required for implanting the staple being limited and with it being possible to control the amount of compression that is applied to the bone portions.

To this end, embodiments of the invention include an instrument for fitting compression staples, the instrument comprising a distal receiver portion for receiving a compression staple that comprises two branches interconnected by a transverse bridge, the instrument being characterized in that it further comprises:
a holder suitable for holding a first zone of the bridge of a staple stationary relative to the receiver portion, while the branches of the staple project distally from the receiver portion, the first zone being selected from two zones consisting respectively in the two end portions of the bridge that are connected to the branches and in the central portion of the bridge; and
deformation tool for deforming a staple from an initial configuration for implanting in a bone towards a compression-applying configuration, said deformation tool being adapted, while the holder hold the first zone of the bridge of the staple stationary relative to the receiver portion, to apply forces to the second zone of the bridge of the staple producing a resultant force in opposition to the force applied to the first zone by the holder.

According to embodiments of the present invention, the staple may be deformed by applying stresses solely to the bridge of the staple, and more precisely via three bearing regions that are distributed along the bridge, e.g. one of its end portions, its central portion, and its opposite end portion. The stresses are applied in opposing manner firstly to the central portion and secondly to the two end portions, so as to cause the staple to deform plastically in controlled manner in order to apply compression to the two bone portions interconnected by the staple. The surgeon can thus control the magnitude of the plastic deformation, it being understood that the compression staple used is made for example of stainless steel or of titanium, which is not possible with staples made of shape memory material, where the magnitude of the compression load is pre-imposed. In addition, by acting solely on the bridge of the staple, the staple can be put into place in a single operation, e.g. by acting on the bridge while the branches of the staple are already fully engaged in the bone, unlike existing equipment that requires the staple to be pushed in with two successive procedures. Positioning the staple in a single movement also makes it possible to control the extent to which the free ends of the branches of the staple project from the cortical bone opposite from that against which the bridge is placed: in order to achieve bone compression under good conditions, it is preferable for the branches of the staple to pass right through the two bone portions that are to be consolidated, and for this to occur before beginning to apply compression, whereas at the end of fitting, it is desirable for the free ends of the branches to project as little as possible from the cortex of said bone portions in order to limit injury to soft tissue, according to embodiments of the present invention.

In practice, embodiments of the invention can operate in two different ways depending on whether the zone of the bridge that is held stationary is its central portion or its two end portions, with deformation stress being applied to the other one of the two zones, the respective specifications for these two embodiments being presented in greater detail below with the help of two examples.

In one embodiment, when the first and second zones of the bridge of the staple correspond respectively to the end portions and to the central portion of the bridge, the deformation tool and the holder are adapted to apply their forces respectively on the two side faces of the bridge. Under such circumstances, deforming stress is applied to the central portion by acting in opposing manner on the sides of the bridge so that the bridge tends to bend in its central portion while remaining overall in a plane that is perpendicular to its branches, thereby causing the branches of the staple to move towards each other, and thus applying compression between the two bone portions in which the branches are inserted.

Such an embodiment may include one or more of the following characteristics, taken in isolation or in any technically feasible combination:
the deformation tool comprises a deformation part movable relative to the receiver portion in a travel direction that, at least when the deformation part is acting on the staple positioned on the receiver portion, extends in a manner that is substantially perpendicular both to the mean plane of the staple and to the longitudinal axis of the bridge of said staple;
the deformation part presents a curved distal end, in particular presenting a V-shape or U-shape extending in the travel direction towards the receiver portion, in such a manner that, while the holder holds the end portions of the bridge of the staple stationary relative to the receiver portion, said end exerts on the central portion of said bridge a thrust force that tends to cause the central portion to bend, in particular to fold said central portion into a V-shape and/or U-shape;
the deformation part is assembled to the receiver portion by an assembly that pivots about an axis relative to which the travel direction is substantially circumferential; and
the holder includes two housings for receiving end portions of the bridge of the staple, said housings being shaped in the receiver portion so that, while the deformation tool is acting on the central portion of the bridge, it guides the relative movement of the respective longitudinal axes of the branches of the staple towards each other, while maintaining said axes mutually parallel, while leaving each branch free to turn about its own axis.

In another embodiment, when the first and second zones of the bridge of the staple correspond respectively to the central portion and to the end portions of the bridge, the deformation tool is adapted to apply its forces on the front face of the bridge that is remote from the branches, the holder preferably being adapted to co-operate with the central portion of the bridge solely on the side of the bridge that faces away from the branches. Under such circumstances, deformation stress is applied to the two end portions by acting in deformation on the front side of the bridge facing away from the branches, such that under the effect of a driving force applied to the end portions, the distal ends of the branches move towards each other, thereby generating a force urging the bone portions in which the branches are inserted towards each other and/or compressing them together.

Such an embodiment may include one or more of the following characteristics, taken in isolation or in any technically feasible combination:
the deformation tool includes a deformation part that is movable in translation relative to the receiver portion, along a translation axis parallel to a mean plane of a staple positioned on the receiver portion and substantially perpendicular to a longitudinal axis of the bridge of said staple;
the deformation part includes a distal portion provided with two side portions each defining a face that is inclined at a non-zero angle relative to the translation axis, each inclined face being adapted, while the holder is holding the central portion of the bridge of the staple stationary relative to the receiver portion, to exert on one of the end portions of said bridge a thrust force tending to cause the branch associated with said end portion to converge towards the other branch;
the deformation part is movable in translation along the translation axis between a first position in which the deformation part releases access to the receiver portion for positioning a staple on the receiver portion, and a second position in which the deformation part is suitable for co-operating with each end portion of the bridge of a staple, while the holder holds the central portion of the staple stationary relative to the receiver portion, the instrument including resilient return means urging the deformation part towards its first position and actuator for driving movement in translation of the deformation part from its first position towards its second position against the return means;
   the actuator may include a lever defining an eccentric cam that co-operates with a proximal portion of the deformation part;
the actuator lever is pivotally mounted relative to the receiver portion to pivot about an axis perpendicular to the axis of movement in translation of the deformation part relative to the receiver portion;
the holder comprises a peg projecting from the receiver portion, the peg being suitable for co-operating with a corresponding housing in the central portion of the bridge of the staple; and
the holder comprises a portion in relief projecting relative to the receiver portion, said portion in relief being suitable for co-operating with a face of the bridge of the staple that faces away from the branches.

An assembly for fitting a compression staple according to embodiments of the present invention includes the compression staple having two branches interconnected by a transverse bridge, and also an instrument as described above.

A method of fitting a compression staple to compress, fuse, join, span, and/or unite two bone portions according to embodiments of the present invention includes:
drilling a bore in each of the two bone portions;
holding a first zone of the bridge of the staple stationary relative to a distal receiver portion forming part of an instrument, the first zone being selected from two zones respectively consisting of the two end portions of the bridge connected to the branches and the central portion of the bridge;
inserting one of the branches of the staple in the bore in one of the bone portions and the other branch in the bore in the other bone portion until the front face of the bridge facing towards the branches comes into contact with the bone portions;
while continuing to hold the first zone of the bridge of the staple stationary, deforming the staple from an initial implantation configuration towards a compression-applying configuration, by applying forces to the second zone of the bridge of the staple producing a resultant force in opposition to the force applied to the first zone for holding it stationary; and
disengaging the receiver portion from the staple.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

The characteristics and advantages of the invention appear in the following description of two embodiments of an instrument and an assembly of the invention for fitting a compression staple, given purely by way of example and with reference to the drawings, in which:

FIG. 1 is an exploded perspective view of a first embodiment of a staple-fitter assembly in accordance with the invention, associated with bone elements to be compressed, according to embodiments of the present invention.

FIG. 2 is an enlarged view of area II of FIG. 1, according to embodiments of the present invention.

FIG. 3 is a perspective view of the staple-fitter assembly of FIG. 1 during fitting of the staple of said assembly, according to embodiments of the present invention.

FIG. 4 is an elevation view of the FIG. 1 bone at the end of the first staple-fitting step, it being observed that the instrument of the staple-fitter assembly is not shown for reasons of visibility, according to embodiments of the present invention.

FIG. 5 is a view analogous to FIG. 2 showing the staple-fitter assembly during a staple-fitting step subsequent to that of FIGS. 3 and 4, according to embodiments of the present invention.

FIG. 6 is a view analogous to FIG. 4 showing the bone and the staple at the end of the subsequent staple-fitting step, according to embodiments of the present invention.

FIG. 7 is a perspective view of a second staple-fitter assembly during a first step of fitting a compression staple in a bone, according to embodiments of the present invention.

FIG. 8 is a partially cut-away enlarged view of area VIII of FIG. 7, according to embodiments of the present invention.

FIG. 9 is a fragmentary section on a larger scale on plane IX of FIG. 7, according to embodiments of the present invention.

FIG. 10 is a fragmentary section on a larger scale on plane X of FIG. 7, according to embodiments of the present invention.

FIG. 11 is a section analogous to FIG. 10, during a second step of fitting the staple in a bone, according to embodiments of the present invention.

FIG. 12 is a view analogous to FIG. 7, during a third step of fitting the staple in a bone, according to embodiments of the present invention.

FIG. 13 is a fragmentary section on a larger scale on plane XIII of FIG. 12, according to embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention and of the appended claims.

### DETAILED DESCRIPTION

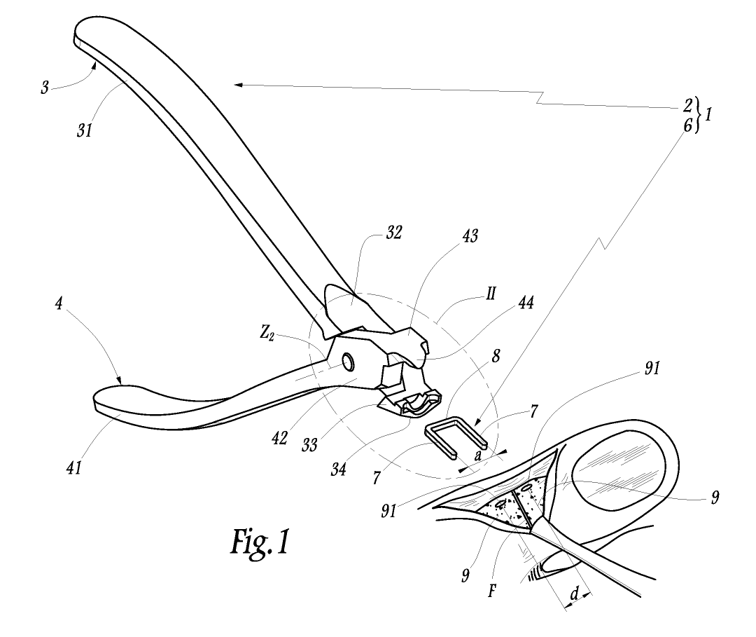

FIG. 1 shows a staple-fitter assembly 1 comprising an instrument 2 and a compression staple 6 for implanting in a bone with the instrument for the purpose of compressing, fusing, joining, spanning, and/or uniting two bone portions 9 that result from a fracture or an osteotomy, according to embodiments of the present invention.

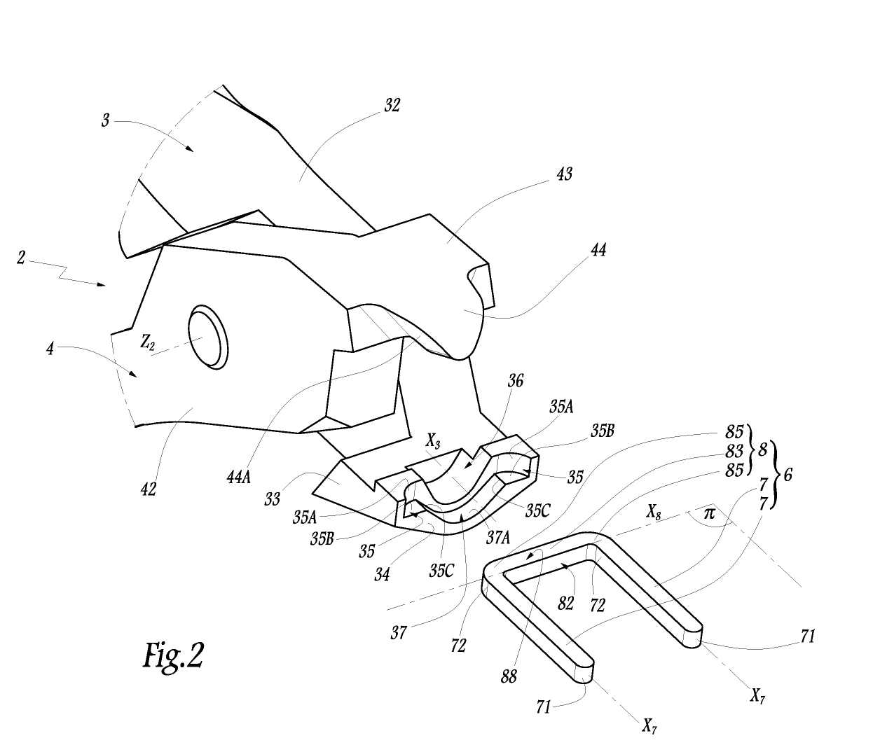

The staple 6 is constituted by two elongate branches 7 and by a transverse bridge 8 interconnecting the proximal ends 72 (FIG. 2) of the two branches. In practice, the staple may be made of stainless steel or of titanium, or indeed, more generally, of any suitable metallic material.

Prior to being implanted in a bone, the staple 6 is in an initial configuration as shown in FIGS. 1 to 3 in which the longitudinal axes X₇ of the two branches 7 are mutually parallel, defining a mean plane π of the staple, and perpendicular to the longitudinal axis X₈ contained in the plane π of the bridge 8 that extends substantially rectilinearly. It will be understood that in this initial configuration, the staple 6 presents an overall structure that is both simple and easy to fabricate.

According to embodiments of the present invention, for the reasons set out below, the staple 6 does not present any sharp edge, in particular along its branches 7. In one embodiment, the cross-section of each branch is not exactly square, in the sense that the four corners of the sections are significantly rounded, typically presenting a radius of curvature of 0.3 millimeters. In practice, to make the edges of the staple less sharp, at the end of fabrication, the staple is subjected to polishing, and in particular to a tumbling method that consists in tumbling the staples together with abrasive particles in a drum.

As shown in FIG. 1, the instrument 2 has two crossed branches 3 and 4 that are hinged to each other about an axis Z₂ perpendicular to the respective longitudinal directions of the branches. The instrument 2 is thus similar to a pair of pliers, according to embodiments of the present invention.

The branch 3 is constituted by a proximal portion 31 enabling the instrument 2 to be held, an intermediate portion 32 mechanically connected to the branch 4 by a hinge pin on the axis Z₂, and a distal portion 33 adapted to receive the staple 6, as explained in detail below. According to such embodiment, the portions 32 and 33 of the branch 3 extend generally rectilinearly one in line with the other along a proximal-distal axis X₃ in FIG. 2.

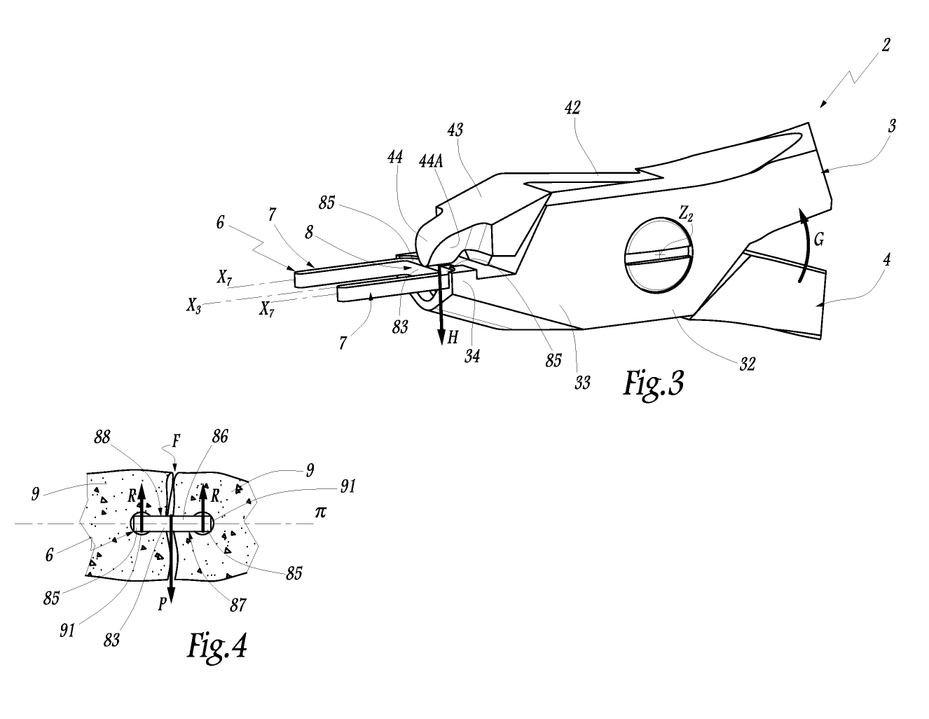

As illustrated in FIG. 2, at its distal end 34, the distal portion 33 is shaped to receive and hold the two end portions 85 of the bridge 8 of the staple 6, while the branches of the staple project distally from the distal end 34, with the respective longitudinal axes X₇ of the branches extending substantially parallel to the axis X₃. For this purpose, in its sides, the distal end 34 defines two respective facing housings 35 shaped to receive in complementary manner the end portions 85 of the staple bridge 8 while the staple is in its initial implantation configuration, as shown in FIG. 3. As shown in FIG. 2, each of these housings 35 is defined firstly by a curved side surface 35A of shape complementary to the corresponding end portion 85 of the bridge 8, more precisely to the corresponding portion of the front face 86 of the bridge remote from the branches 7, and secondly by a plane bottom surface 35B that forms a bearing surface for the corresponding end portion 85 of the bridge, more precisely for the corresponding portion of one of the side faces of the bridge 8, as identified by reference 87 in FIG. 4, according to embodiments of the present invention.

Between the two housings 35, the distal end 34 of the portion 33 of the branch 3 is recessed, as shown in FIG. 2, forming a cavity 36 that is open to face in the same direction as the bottom surfaces 35B, while said cavity is closed in the opposite direction. As an optional arrangement, the cavity 36 includes a housing 37 interconnecting the surfaces 35B of the housings 35 via a concave surface 37A set back below the housings 35. This surface 37A is generally V-shaped with a rounded tip.

The branch 4 of the instrument 2 is likewise constituted by a proximal portion 41 for holding the instrument 2, an intermediate portion 42 assembled to the portion 32 of the branch 3 in a hinged manner, and a distal portion 43 adapted to apply deforming forces to the staple 6. For this purpose, the side of the distal end 44 of said portion 43 that faces towards the distal portion 33 of the branch 3 is given a rounded shape presenting a convex surface 44A. In the example under consideration, and in section across the branch 4, this convex surface 44A presents a V-shaped profile with a rounded tip as illustrated in FIGS. 2 and 3. As a result, the rounded distal end 44 is shaped to be received in the cavity 36 of the distal end 34 of the branch 3 in complementary manner, while leaving between said surface 44A and the surface 37A of the housing 37 a gap that is sufficient to place between them the central portion 83 of the bridge 8, as considered between its opposite side faces 87 and 88.

As illustrated in FIG. 1, other structural characteristics of the instrument 2 are described below when describing an example of how the instrument may be used to fit the compression staple 6 for the purpose of joining and compressing (e.g. pinching together) the two bone portions 9. By way of example, consideration is given to two bone portions 9 forming parts of a phalanx or a metatarsus that has been fractured or subjected to osteotomy, with the anatomically lower face of said phalanx or metatarsus facing downwards in FIGS. 4 and 6, while its anatomically upper surface faces upwards in these figures.

Initially, a bore 91 is drilled in each of the two bone portions 9 on either side of the site F of the fracture or the osteotomy of the bone. When the bone portions face each other without compression, as shown in FIG. 1, the bores 91 are thus spaced apart from each other by a distance d equal to the distance a between the axes of the branches 7 of the staple 6 in an initial configuration for implantation.

Advantageously, and for reasons that are explained below, the bores 91 are not drilled in the anatomically upper faces of the bone portions 9, but in one of the anatomical side faces of the bone, in particular its side face that is easier for the surgeon to reach.

Before, simultaneously with, or after drilling the bores 91, the staple 6 in the initial configuration for implantation is positioned on the distal end 33 of the branch 3. For this purpose, the distal ends 33 and 43 of the branches 3 and 4 are moved apart from each other, by moving their proximal portions 31 and 41 apart by pivoting about the axis Z₂. The bridge 8 of the staple is then put into place in such a manner that its end portions 85 are received in respective ones of the housings 35, with the side face 87 and the front face 86 of the bridge bearing respectively against the surfaces 35B and against the surfaces 35A of these housings 35 at the bridge portions 85. As can be seen in FIG. 3, the branches 7 then project distally from the distal portion 33.

In order to hold the staple 6 stationary relative to the branch 3, the distal portions 33 and 43 of the branches 3 and 4 are moved progressively towards each other, by moving their proximal portions 31 and 41 in corresponding manner by pivoting about the axis Z₂, until the distal end 44 of the portion 43 comes into contact against the side face 88 of the bridge 8 in the central portion 83 thereof, but without stressing there against. The instrument 2 is then in the configuration of FIG. 3.

Once the staple 6 is held in this way relative to the instrument 2, the branches 7 are inserted into the bores 91. To do this, the distal end 71 of each branch is positioned facing a respective one of the bores 91, and then a thrust force is exerted via the branch 3 against the end portions 85 of the bridge 8 via the surfaces 35A of the housings 35. Each branch 7 then penetrates into the corresponding bore 91 until the front face 82 of the bridge 8 that faces towards the branches 7 comes into contact against the anatomical side surfaces of the bone portions 9, as shown in FIG. 4.

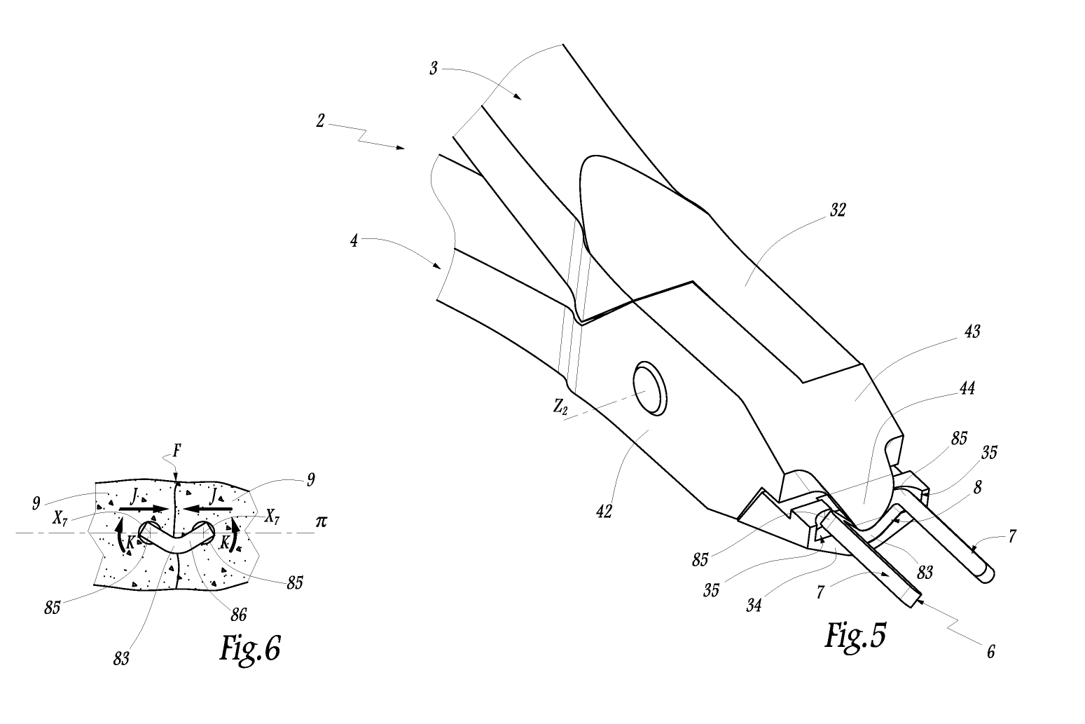

Once the branches 7 have been inserted (e.g. maximally) into the bores 91, the staple 6 is deformed so as to put the bone portions 9 into compression. For this purpose, the instrument 2 is actuated so as to bring the proximal portions 31 and 41 of the branches 3 and 4 closer together. By tilting about the axis Z₂ of the hinge pin, as represented by arrow G in FIG. 3, the distal portions 33 and 43 of the branches move towards each other, the distal end 44 of the branch 4 seeking to penetrate into the cavity 36 in the distal end 34 of the branch 3. The surface 44A of the end 44 exerts a thrust force P on the central portion 83 of the bridge 8, said thrust force P, shown in FIG. 4, being directed in a direction H that is substantially circumferential about the axis Z₂. This direction H corresponds to the direction of local relative movement between the distal ends 34 and 44 of the branches 3 and 4, as shown in FIG. 3. Simultaneously, the end portions 85 of the bridge 8 remain stationary relative to the distal portion 33, each being subjected to a reaction force R that is exerted by the bottom surfaces 35B of the housings 35 and that is directed in the opposite direction to the thrust force P, as shown in FIG. 4. Thus, the thrust force P exerted on the central portion 83 and the reaction forces R exerted on the end portions 85 oppose one another, with their magnitudes being such that the combination of these forces causes the staple 6 to be deformed from its initial configuration for implantation to a configuration for applying compression, as shown in FIGS. 5 and 6.

Insofar as the travel direction H (FIG. 2) of the distal end 44 of the branch 4 relative to the distal portion 33 of the branch 3 extends perpendicularly both to the mean plane π of the staple 6 and to the longitudinal axis X₈ of the bridge 8 of the staple, the surface 44A of the end 44 causes the central portion 83 of the bridge 8 to bend with progressive plastic deformation of said central portion 83, so that it takes up a shape that is substantially complementary to the rounded shape of said surface 44A, and centered on the travel direction H. Given the V-shaped profile of the surface 44A under consideration, the central portion 83 of the bridge 8 is progressively folded into a V-shape until it reaches the configuration shown in FIGS. 5 and 6. When it has this bent shape, the bridge 8 occupies a plane that is perpendicular to the mean plane π.

It will be understood that the bending of the central portion 83 causes the end portions 85 to move towards each other, and thus causes the branches 7 to move towards each other. In other words, the distance between the axes decreases, thereby generating forces J causing the bone portions 9 to approach each other, as shown in FIG. 6. In the site F, the facing surfaces of these bone portions 9 are pressed one against the other, with a certain amount of pressure. Since the central portion 83 of the bridge 8 is deformed plastically, the compression-applying configuration of the staple 6 is particularly stable over time, according to embodiments of the present invention.

In practice, the relative movement of the end portions 85 of the bridge 8 towards each other is guided by the bottom surfaces 35B of the housings 35, thus enabling the branches 7 to be kept parallel in the mean plane π of the staple 6. Since the branches 7 are kept parallel to each other in the configuration for putting the staple 6 into compression, these two branches contribute over their entire length to the compression forces J, according to embodiments of the present invention. The facing ends 35C of the surfaces 35B of the two housings 35 may be shaped to allow the branches 7 to turn freely about their respective axes X₇, as represented by arrows K in FIG. 6, in order to improve the way in which the branches 7 are kept parallel as they move towards each other. The edges of these branches may be rounded, thus facilitating their turning inside the bores 91, according to embodiments of the present invention.

After the staple 6 has been deformed in this way, the branch 4 is moved away from the branch 3 and then the instrument 2 is removed. Beforehand, if necessary, a small amount of impacting may be performed on the bridge 8 of the staple 6 in order to fully absorb any residual clearance between the face 82 of the bridge and the side faces of the bone portions 9 that might appear while bending the central portion 83.

In this way, the instrument 2 enables compression to be applied to the bone portions 9 while the staple 6 is already maximally engaged in the bone, e.g. when the bridge 8 of the staple is in contact with the surface of the bone. The retention by the distal portion 33 of the branch 3, and the deformation by the initial portion 43 of the branch 4 are not obtained by acting on the side of the bridge that faces towards the branches 7, but solely by acting on the side faces 87 and 88 in the end portions 85 and the central portion 83 of the bridge, according to embodiments of the present invention. Furthermore, the instrument 2 permits the compression force applied to the bone portions 9 by the staple 6 to be controlled, since the magnitude of the thrust force P is directly under the control of the surgeon acting on the branches 3 and 4. Thus, after applying a first magnitude of deformation on the staple, the surgeon can apply a greater magnitude to deform the staple more, e.g. to cause its central portion 83 to bend more and thus to reinforce the compression forces J, should that be necessary. At the most, the central portion 83 can thus be bent until the side face 88 of the bridge 8 is pressed in complementary manner against the surface 37A of the housing 37. Furthermore, regardless of how strongly the surgeon moves the proximal portions 31 and 41 of the branches 3 and 4 towards each other, this ensures that there is no risk of the surgeon breaking the bridge 8, according to embodiments of the present invention.

It should be observed that the space occupied by the bridge 8, in particular when its central portion 83 is bent, is particularly small, according to embodiments of the present invention. The risk of discomfort under the skin is therefore limited, as is the risk of tissue close to the staple 6 being injured. Furthermore, depending on which side face 87 or 88 of the bridge 8 the surgeon decides to apply the thrust force P, the central portion 83 is bent exclusively to one side or to the other side of the mean plane π of the staple 6. Thus, in the embodiment shown in FIGS. 4 and 6, the central portion 83 is bent downwards, in an anatomical zone where the tissue is a priori less sensitive and less exposed than tissue on the anatomically upper side of the consolidated phalanx or metatarsus.

The instrument 2 and the associated staple 6 may include one or more alterations and/or variations, according to embodiments of the present invention. For example:
as well as or instead of using the distal portion 43 of the branch 4 to hold the staple 6 stationary in position on the distal portion 33 of the branch 3, the instrument 2 may include an autostatic system preventing the staple from separating from the instrument while its branches 7 are being put into place in the bores 91 in the bone fragments; such an autostatic system may include in particular a mechanism for preventing the branches of the instrument from moving when they are configured to hold the staple strongly but without deforming it;
in order to facilitate turning of the branches 7 about their own axes in the bores 91 in the bone fragments, these branches or indeed the entire staple 6, may present a cross-section having a circular outline;
profiles other than a V-shape with a rounded point may be envisaged for the distal end 44 of the branch 4, in particular a profile that is continuously arcuate so as to cause the central portion 83 of the bridge 8 to bend with continuous curvature that is substantially in the form of a circular arc and/or a U-shape;
with more complex arrangements of the staple 6 and/or of the distal portion 33 of the branch 3, it is possible to limit or even avoid the branches 7 turning about their own axes, providing it is made possible for the end portions 85 of the bridge 8 to move in translation towards each other in the mean plane π of the staple while the central portion 83 is being deformed; and/or
instead of assembling the branch 4 pivotally on the branch 3, the distal portion 43 of the branch 4 may include more elaborate arrangements for enabling it to be moved in translation in the travel direction H of its distal end 44.

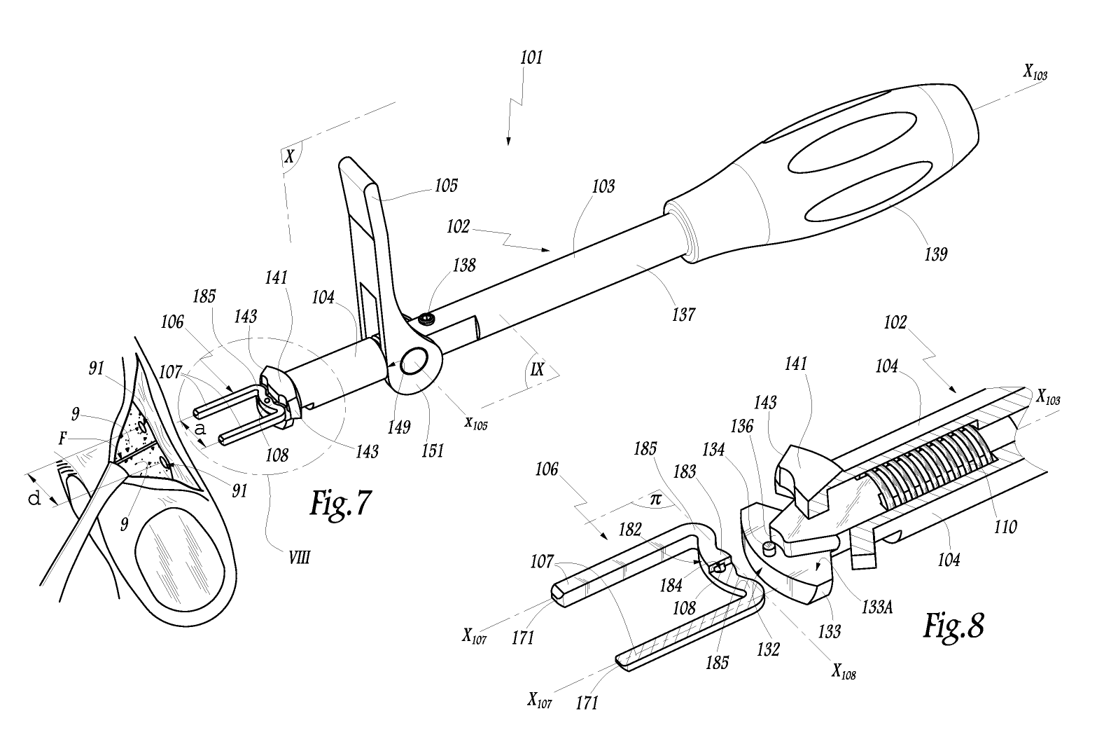

The staple-fitter assembly 101 shown in FIG. 7 comprises an instrument 102 and a compression staple 106 for implanting in a bone by means of the instrument 102 in order to join and compress two portions 9 of the bone after a fracture or an osteotomy, according to embodiments of the present invention.

The staple 106 has two branches 107 of elongate shape and a transverse bridge 108 interconnecting the two branches. In this embodiment, the staple is made of stainless steel. In a variant, the staple could be made of any other suitable material, e.g. out of titanium.

Prior to being implanted in a bone, the staple 106 is in an initial implantation configuration, as shown in FIGS. 7 to 11, in which the longitudinal axes X₁₀₇ of the two branches 107 are substantially parallel to each other and perpendicular to the longitudinal axis X₁₀₈ of the bridge 108. In this initial implantation configuration, the bridge 108 is curved towards the branches 107. As illustrated in FIG. 8, a mean plane of the staple 106 defined by the longitudinal axis X₁₀₇ of each of the branches 107 and the longitudinal axis X₁₀₈ of the bridge 108 is referenced π.

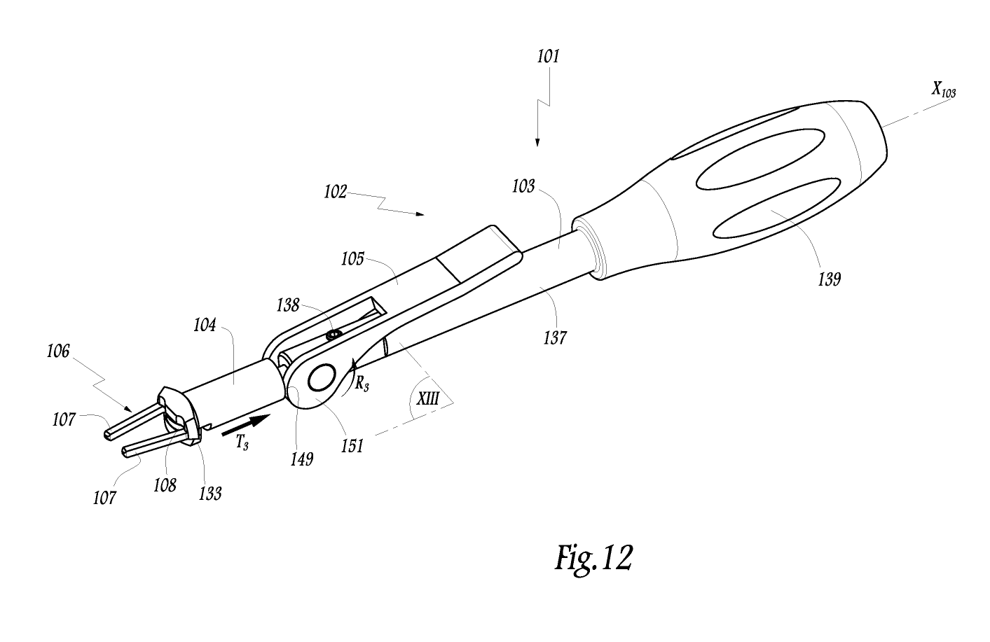

Because of the mechanical properties of the material from which it is made, the staple 106 is suitable for being deformed from its initial implantation configuration to a compression-applying configuration, as shown in FIGS. 12 and 13, in which the branches 107 converge while remaining in the mean plane π. By converging in this way, when in the implanted configuration with each branch of the staple 106 in one of the bone portions, the branches are suitable for exerting a compression force pressing the bone portions against each other. In this compression-applying configuration, the bridge 108 is substantially rectilinear, according to embodiments of the present invention.

The staple 106 is deformed from its initial implantation configuration to its compression-applying configuration by applying a thrust force P on each end portion 185 of the bridge 108 that is connected to a branch 107, as shown in FIG. 13, the force P being directed towards the end 171 of the other branch 107 that is distal relative to the bridge 108, while a central portion 183 of the bridge 108 is held stationary.

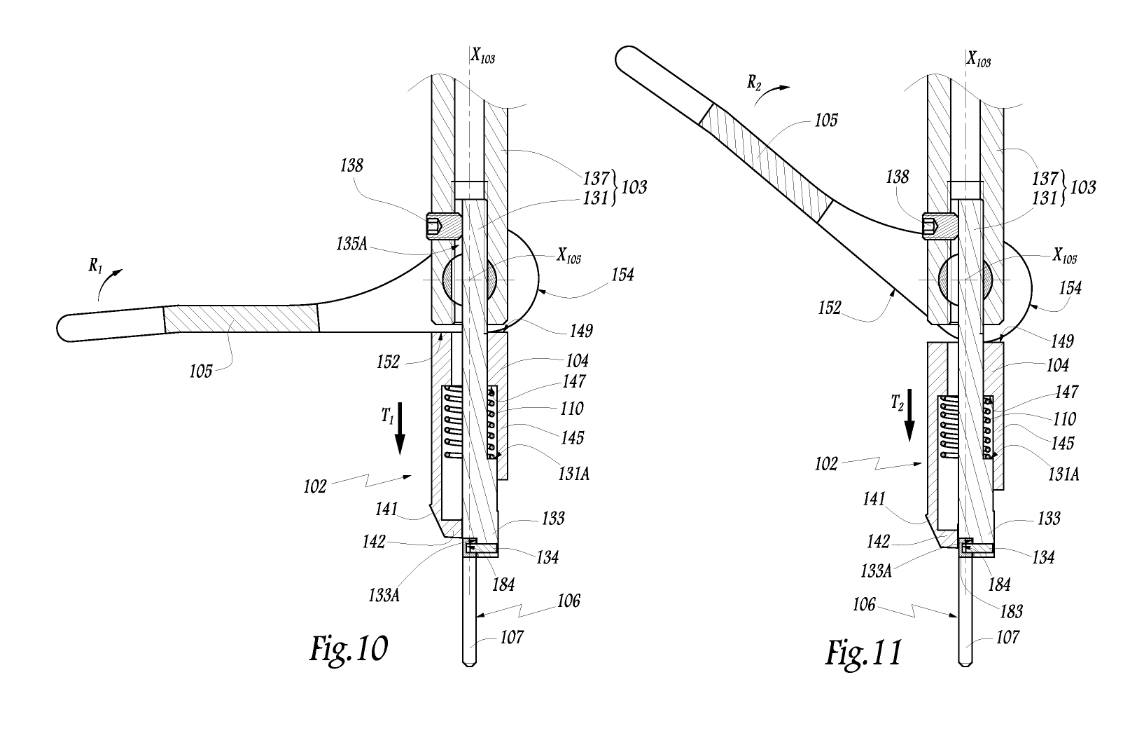

The instrument 102 for fitting the staple 106 in the bone portions comprises a main body 103 of elongate shape having a longitudinal axis referenced X₁₀₃. The main body 103 has a distal rod 131 and a tubular element 137 engaged around a proximal end portion 135 of the rod 131. As shown in FIGS. 8 and 10, the proximal portion 135 of the rod 131 is generally cylindrical, of circular section that is truncated at a flat 135A. The rod 131 and the tubular element 137 are held stationary relative to each other by means of a screw 138 that bears against the flat 135A, the length of the proximal portion 135 that is received in the tubular element 137 being adjustable and set by means of the screw 138. A proximal end portion 139 of the tubular element 137 forms a handle for the instrument 102, while a distal end portion 133 of the rod 131, of a flat shape, is shaped to receive the staple 106 for implanting in a bone.

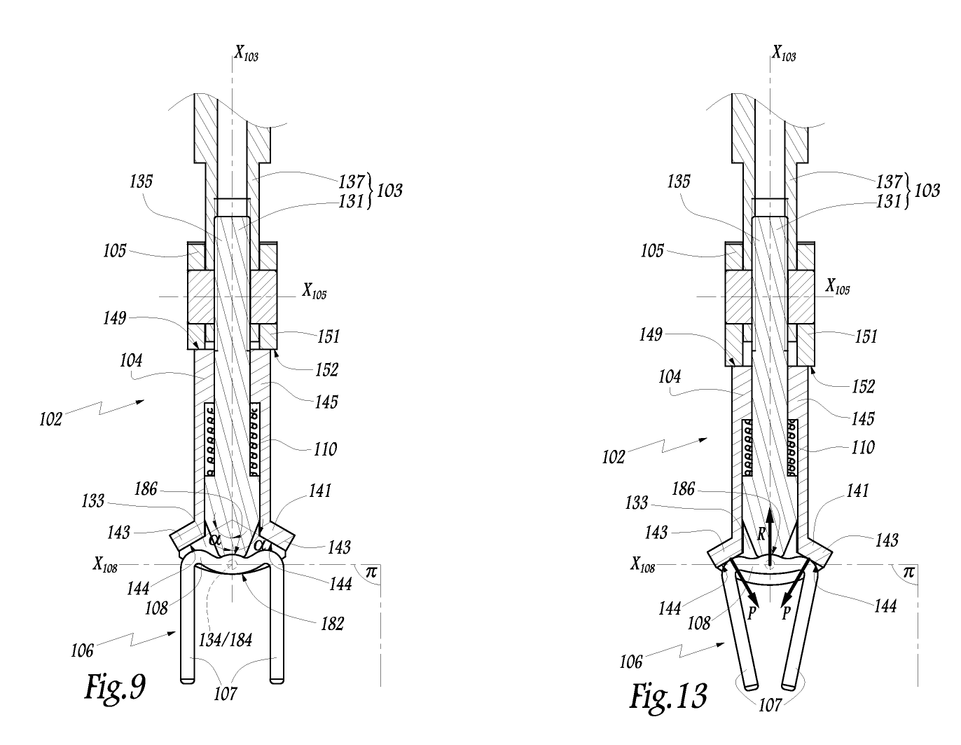

The staple 106 is designed to be positioned on the staple receiver portion 133 with the bridge 108 received bearing against a side face 133A of the receiver portion 133 and with the branches 107 projecting distally from the receiver portion 133, as can be seen in particular in FIGS. 9 and 10. In order to guarantee that the staple 106 is accurately positioned on the receiver portion 133, the portion 133 includes a peg 134 projecting from the face 133A, arranged in the vicinity of the distal end 132 of the rod 131 and designed to be inserted in a corresponding housing 184 drilled in the central portion 183 of the bridge 108 of the staple, according to embodiments of the present invention. The receiver portion 133 also includes a portion in relief 136 projecting from the face 133A and suitable for co-operating with a front face 186 of the bridge 108 of the staple, facing away from the branches 107, in the central portion 183 of the bridge 108 when the peg 134 is received in the orifice 184. In its configuration with the bridge 108 of the staple 106 positioned relative to the peg 134 and to the portion in relief 136, the longitudinal axis X₁₀₃ of the main body 103 of the instrument is parallel to the mean plane π of the staple 106, and substantially perpendicular to the longitudinal axis X₁₀₈ of the bridge 108 of the staple.

The instrument 102 also has a part 104 for deforming the staple 106 from its initial implantation configuration towards its compression-applying configuration. The deformation part 104 is movable in translation relative to the rod 131 parallel to the longitudinal axis X₁₀₃ between a first position, visible in FIGS. 7 to 9, in which the deformation part 104 leaves free access to the receiver portion 133 for positioning the staple 106, and a second position, visible in FIGS. 12 and 13, in which the deformation part 104 is suitable for cooperating with each end portion 185 of the bridge 108 of the staple 106 positioned on the receiver portion 133 by means of the peg 134 and the portion in relief 136, according to embodiments of the present invention.

The deformation part 104 has a cylindrical proximal portion 145 that guides the deformation part 104 in movement in translation relative to the rod 131 along the axis X₁₀₃. The deformation part 104 also has a distal cap 141 arranged solely facing the face 133A of the receiver portion 133. As illustrated in FIG. 9, the cap 141 is provided with two side tabs 143 each defining an inclined face 144 that is at an angle α of about 60° relative to the translation axis X₁₀₃. In a variant, the angle α may have a value other than 60°, preferably a value lying in the range about 30° to about 70°. In the second position of the deformation part 104, as can be seen in FIGS. 12 and 13, the two side tabs 143 are suitable for cooperating simultaneously with the two end portions 185 of the bridge 108 of the staple 106 in position on the receiver portion 133.

The profile of the cap 141 is designed in such a manner that when the deformation part 104 is in an intermediate position between its first and second positions, as can be seen in FIG. 11, the central portion 183 of the bridge 108 of the staple is held stationary relative to the receiver portion 133, between the face 133A of the receiver portion 133 and the central portion 142 of the cap 141. In addition, in the second position of the deformation part 104, the face 144 of each side tab 143 is suitable for exerting a thrust force P on an end portion 185 of the bridge 108, as shown in FIG. 13. Because of the inclination of each face 144 of each side tab 143 at an angle α relative to the translation axis X₁₀₃, the force P exerted on an end portion 185 adjacent to a branch 107 is directed towards the distal end 171 of the other branch 107. In the second position of the deformation part 104, a thrust force P is applied to each end portion 185 of the staple 106, while the central portion 183 of the bridge 108 of the staple is prevented from moving relative to the receiver portion 133, such that the staple 106 tends to deform from its initial implantation configuration towards its compression-applying configuration, in which the branches 107 converge.

A compression spring 110 is located between a shoulder 131A of the rod 131 and an internal radial shoulder 147 of the proximal portion 145 of the deformation part 104. The spring 110 urges the deformation part 104 elastically towards its first position, in which the cap 141 is offset on the proximal side parallel to the axis X₁₀₃ relative to the face 133A of the receiver portion 133.

To actuate movement in translation of the deformation part 104 from its first position towards its second position, against the spring 110, the instrument 102 includes a lever 105 pivotally mounted on the distal portion 135 of the rod 131 about an axis X₁₀₅ perpendicular to the translation axis X₁₀₃. The lever 105 defines an eccentric cam 151 for co-operating with the proximal end 149 of the deformation part 104. The lever 105 is suitable for being moved from an initial position in which a small-diameter cam surface 152 of the eccentric cam 151 co-operates with the proximal end 149 of the deformation part 4, thereby forming an abutment for holding the deformation part 104 in its first position, and a maximally actuated position in which a cam surface 145 of the eccentric cam 151 having a diameter greater than the diameter of the cam surface 152 cooperates with the proximal end 149 of the deformation part 104 and holds the deformation part 104 in its second position. The diameter of the peripheral surface of the eccentric cam 151 increases continuously between its surfaces 152 and 154, according to embodiments of the present invention.

A method according to embodiments of the present invention includes fitting the compression staple 106 of the staple-fitter assembly 101 with the instrument 102 for the purpose of joining and compressing two bone portions 9 of a bone that has fractured or that has been subjected to an osteotomy, e.g. a phalanx or a metatarsus.

Firstly, a bore 91 is drilled in each of the two bone portions 9 on either side of the site F of the bone fracture or osteotomy. Advantageously, in the configuration in which the bone portions 9 touch each other without compression, as shown diagrammatically in FIG. 7, the bores 91 are spaced apart from each other by a distance d equal to or slightly greater than the distance a between the axes of the branches 107 when the staple 106 is in the initial implantation configuration, with the branches 107 parallel to each other.

Before, simultaneously with, or after drilling the bores 91, the staple 106 in its initial implantation configuration is positioned on the receiver portion 133 of the instrument 102. For this purpose, where necessary, the instrument 102 is set into its configuration that is shown in FIGS. 7 to 10 with the lever 105 in its initial position and the deformation part 104 in its first position spaced apart from the receiver portion 133. The bridge 8 of the staple 6 is then placed against the face 133A of the receiver portion 133 so that the housing 184 in the central portion 183 of the bridge 108 cooperates with the peg 134 and the face 186 of the bridge 108 cooperates with the portion in relief 136. Once the staple 106 is positioned in this way on the receiver portion 133, the branches 107 project distally from the receiver portion 133, as can be seen in particular in FIGS. 7 and 10, according to embodiments of the present invention.

The central portion 183 of the bridge 108 of the staple is then held stationary relative to the receiver portion 133 by actuating a first movement in translation T₁ of the deformation part 104 along the axis X₁₀₃ from its first position towards its intermediate position in which the central portion 142 of the cap 141 faces the central portion 183, the portion 183 thus being held between the cap 141 and the face 133A. This first movement in translation T₁ of the deformation part 104 is actuated by turning the lever 105 through a first angle in the direction of arrow R₁ in FIG. 10. In combination with the peg 134 and the portion in relief 136, the deformation part 104 then prevents the central portion 183 from moving relative to the receiver portion 133, with the staple 106 still being in its initial implantation configuration.

Once the central portion 183 of the bridge 108 is held in this way relative to the receiver portion 133, the branches 107 of the staple 106 are inserted into the bores 191. In particular, the distal end 171 of each branch 107 is positioned facing a respective bore 191 and a thrust force is exerted on the proximal portion 139 of the main body 103 so that each branch 107 penetrates into the corresponding bore 191 until a front face 182 of the bridge 108 that faces towards the branches 107 comes into contact with the surfaces of the bone portions 9.

Once the branches 107 have been maximally inserted in the bores 91, the staple 106 is deformed to put the bone portions 9 into compression. For this purpose, the deformation part 104 is actuated to perform a second movement in translation T₂ along the axis X₁₀₃ from its intermediate position towards its second position as shown in FIGS. 12 and 13. This second movement in translation T₂ is actuated by turning the lever 105 in the direction of arrow R₂ in FIG. 11. In this second position of the deformation part 104, the faces 144 of the side tabs 143 exert a thrust force P on each end portion 185 of the bridge 108, the force being directed towards the distal end 171 of the other branch 107, while the central portion 183 of the bridge 108 of the staple remains stationary relative to the receiver portion 133. In particular, the peg 134 then exerts a reaction force R on the central portion of the bridge 108, this force R being directed away from the branches 107. The combination of the thrust forces P exerted on the end portions 185 and the traction force R exerted on the central portion 183 gives rise to the desired deformation of the staple 106 from its initial implantation configuration towards its compression-applying configuration, as shown in FIG. 13.

The movement of the distal ends 171 of the branches 107 towards each other during deformation of the staple 106 then generates a force urging the bone portions 9 towards each other, thereby enabling the surfaces of the two bone portions in the site F to be pressed one against the other with a certain amount of pressure. Because of the mechanical properties of the material constituting the staple 106, the branches 107 continue to converge in spite of the reaction force exerted by the wall of each bore 91 against the corresponding branch.

After the staple 106 has been deformed in this way, the receiver portion 133 of the instrument 102 is separated from the staple 106 by actuating the deformation part 104 to move in translation T₃ along the axis X₁₀₃ from its second position towards its first position so as to release the bridge 108 from the cap 141, and then by removing the staple from the peg 134. The movement in translation T₃ is actuated by turning the lever 105 in the direction of arrow R₃ in FIG. 12, e.g. in the direction opposite to that of arrows R₁ and R₂.

According to such embodiment, the staple-fitter assembly 101 enables compression to be applied to the bone portions 9 while the staple 106 is (e.g. maximally) engaged in the bone, e.g. while the bridge 108 of the staple is in contact with the surface of the bone. The mechanism for preventing the central portion 183 of the bridge of the staple from moving relative to the receiver portion does not act on the side of the bridge that faces towards the branches 107, and the tabs 143 of the deformation part 104 cooperate with the end portions 185 solely on the side of the bridge that is remote from the branches. Under such circumstances, it is possible to deform the staple 106 while it is fully impacted into the bone, thereby eliminating the need to perform two impacting operations in succession on the staple, one before and the other after deformation of the staple. This makes the staple easier to implant, and significantly reduces the time required for implanting it, according to embodiments of the present invention.

Furthermore, the instrument 102 enables the compression load applied to the bone portions 9 by the compression staple 106 to be controlled. It is possible to apply a controlled amount of thrust force P on the end portions 185 of the bridge 108 of the staple so as to generate an appropriate amount of deformation of the staple. In particular, the force P may be adjusted by varying the angle of rotation R₂ of the lever 105, thus enabling the deformation part 104 to be driven in translation to a greater or lesser extent towards the bridge of the staple positioned on the receiver portion 133. This controlled deformation of the staple 106, and indeed that of the staple 6, differs from the deformation that is obtained with known staple-fitter assemblies that make use of compression staples made of shape memory material. For such staples made of shape memory material, the compression load applied to the bone portions for consolidating is imposed by the characteristics of the shape memory material, and there is no way of controlling it while the staple is being implanted. In addition, a compression staple of a staple-fitter assembly according to an embodiment of the present invention, e.g. made of stainless steel or of titanium, is less likely to give rise to allergic reactions than is a staple made of a shape memory material, and it does not put temperature constraints on its storage and its use. A compression staple of a staple-fitter assembly according to an embodiment of the invention, e.g. made of stainless steel or of titanium, can also be fabricated easily, with limited fabrication costs.

Embodiments of the present invention include various alterations and/or variations of the instrument 102 and the associated staple 106. For example:
the mechanism for holding the central portion of the bridge of a compression staple stationary may be of a form other than that described above; by way of example, such mechanism may be constituted by a distal end of the instrument forming a clamp suitable for engaging the central portion of the bridge of the staple, on the side of the bridge that is remote from the branches of the staple;
similarly, it is possible to have a mechanism for deforming the staple other than a part that is movable in translation, e.g. a system of pivotally-mounted clamps that can be moved down against each end portion of the bridge of the staple in the configuration where the staple is held relative to the receiver portion; under such circumstances, movement in translation of the part can be equally well-actuated by a structure other than a lever forming an eccentric cam; in particular, the deformation part may be moved in translation by means of a screw system or any other suitable device;
the deformation part 104 may also include a shape other than that described above; the proximal portion of said part need not be cylindrical, with guidance of the deformation part in translation along the axis of the instrument then being provided by any other known guidance mechanism; the distal cap of the deformation part may also present a profile that is different from that described and shown, providing it performs the desired function of deforming a staple that is held stationary relative to the receiver portion, according to embodiments of the present invention; and/or
the compression staple of a staple-fitter assembly according to an embodiment of the invention may present a shape other than shown in the figures; in particular, the bridge 108 may be rectilinear in shape in the initial implantation configuration of the staple, instead of being curved beside the branches 107, it being observed that a curved shape for the bridge beside the branches nevertheless encouraged a low and compact profile for the staple against the surface of the bone when the staple is in its implanted configuration, by limiting the space occupied by the bridge, which tends to deform away from the branches under the combined effects of the thrust forces P applied to the end portions of the bridge and the reaction force R applied to the central portion of the bridge, with such a compact configuration for the staple on the surface of the bone limiting the risk of discomfort under the skin and of injury to tissue in the vicinity of the staple.

Some embodiments of the present invention include one or more alterations and/or variations that applicable to staple 6 and staple 106. For example:
the branches 7 or 107 may present lengths that are different; and/or
the staple 6 or 106 may be provided with a mechanism for holding the branches 7 or 107 in an anchored configuration in the bone portions so as to avoid any escape of the staple from the bone; such anchoring mechanism may be formed by teeth or grooves formed on an outside or inside face of the branches 7 or 107, or indeed by catch members, preferably arranged in the vicinity of the distal or proximal ends of the branches.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. For example, while the embodiments described above refer to particular steps, the scope of this invention also includes embodiments having different combinations of steps and embodiments that do not include all of the described steps. Accordingly, the scope of the present invention is intended to embrace all such alternatives, modifications, and variations as fall within the scope of the claims, together with all equivalents thereof.

What is claimed is:

1. An instrument for fitting compression staples, the instrument comprising:
a distal receiver portion for receiving a compression staple that includes two branches interconnected by a transverse bridge;
a holder suitable for holding a first zone of the bridge of the staple stationary relative to the receiver portion while the branches of the staple project distally from the receiver portion, the first zone selected from the group consisting of two end portions of the bridge that are connected to the branches and a central portion of the bridge; and
a deformation tool for deforming a staple from an initial configuration for implanting in a bone towards a compression-applying configuration, said deformation tool being adapted, while the holder holds the first zone of the bridge of the staple stationary relative to the receiver portion, to apply forces to the second zone of the bridge of the staple producing a resultant force that opposes the forces applied to the first zone by the holder.

2. The instrument according to claim 1, wherein the first and second zones of the bridge of the staple correspond respectively to the two end portions and to the central portion of the bridge, the deformation tool and the holder are adapted to apply their forces respectively on two side faces of the bridge.

3. The instrument according to claim 2, wherein the deformation tool comprises a deformation part movable relative to the receiver portion in a travel direction that, when the deformation part is acting on the staple positioned on the receiver portion, extends in a manner that is substantially perpendicular both to a mean plane of the staple and to a longitudinal axis of the bridge of said staple.

4. The instrument according to claim 3, wherein the deformation part includes a V-shape extending in the travel direction towards the receiver portion, in such a manner that, while the holder holds the end portions of the bridge of the staple stationary relative to the receiver portion, said distal end exerts on the central portion of said bridge a thrust force that tends to cause the central portion to bend, in particular to fold said central portion into a V-shape.

5. The instrument according to claim 3, wherein the deformation part is assembled to the receiver portion by an assembly that pivots about an axis relative to which the travel direction is substantially circumferential.

6. The instrument according to claim 2, wherein the holder includes two housings for receiving end portions of the bridge of the staple, said housings being shaped in the receiver portion such that, while the deformation tool is acting on the central portion of the bridge, the housings guide the relative movement of respective longitudinal axes of the branches of the staple towards each other while maintaining said axes mutually parallel, and while leaving each branch free to turn about its own axis.

7. The instrument according to claim 1, wherein, when the first and second zones of the bridge of the staple correspond respectively to the central portion and to the end portions of the bridge, the deformation tool is adapted to apply forces on the front face of the bridge that is remote from the branches, and wherein the holder is adapted to cooperate with the central portion of the bridge solely on the side of the bridge that faces away from the branches.

8. The instrument according to claim 7, wherein the deformation tool comprises a deformation part that is movable in translation relative to the receiver portion along a translation axis parallel to a mean plane of a staple positioned on the receiver portion and substantially perpendicular to a longitudinal axis of the bridge of said staple.

9. The instrument according to claim 8, wherein the deformation part includes a distal portion provided with two side portions each defining a face that is inclined at a non-zero angle relative to the translation axis, each inclined face being adapted, while the holder is holding the central portion of the bridge of the staple stationary relative to the receiver portion, to exert on one of the end portions of said bridge a thrust force tending to cause the branch associated with said end portion to converge towards the other branch.

10. An assembly for fitting a compression staple, the assembly comprising a compression staple having two branches interconnected by a transverse bridge, and further comprising the instrument according to claim 1.

11. A method of fitting a compression staple to compress two bone portions, the staple including two branches interconnected by a transverse bridge, the method comprising:
drilling a bore in each of the two bone portions;
holding a first zone of the bridge of the staple stationary relative to a distal receiver portion forming part of an instrument, the first zone being selected from the group consisting of two end portions of the bridge connected to the branches and a central portion of the bridge;
inserting one of the branches of the staple in the bore in one of the bone portions and the other branch in the bore in the other bone portion until a front face of the bridge facing towards the branches comes into contact with the bone portions;
while continuing to hold the first zone of the bridge of the staple stationary, deforming the staple from an initial implantation configuration towards a compression-applying configuration by applying forces to the second zone of the bridge of the staple producing a resultant force that opposes the forces applied to the first zone by the instrument; and
disengaging the receiver portion from the staple.

12. The method according to claim 11, further comprising:
applying the forces to the first and second zones on two side faces of the bridge when the first and second zones of the bridge of the staple correspond respectively to the end portions and to the central portion of the bridge.

13. The method according to claim 11, further comprising:
extending a deformation part relative to the receiver portion in a travel direction that is substantially perpendicular both to a mean plane of the staple and to a longitudinal axis of the bridge of the staple.

14. An instrument comprising:
a distal receiver portion configured to receive a compression staple that includes two branches interconnected by a transverse bridge;
a holder configured to hold a first zone of the bridge of a staple stationary relative to the receiver portion while the branches of the staple project distally from the receiver portion, the first zone being either two end portions of the bridge that are connected to the branches or the central portion of the bridge; and
a deformation mechanism configured to deform a staple from an initial configuration for implanting in a bone to a compression-applying configuration;
said deformation mechanism further configured, while the holder holds the first zone of the bridge of the staple stationary relative to the receiver portion, to apply forces to the second zone of the bridge of the staple producing a resultant force that opposes the forces applied to the first zone by the holder.

15. The instrument according to claim 14, wherein when the first and second zones of the bridge of the staple correspond respectively to the end portions and to the central portion of the bridge, and wherein the deformation mechanism and the holder are further configured to apply their forces respectively on two side faces of the bridge.

16. The instrument according to claim 15, wherein the deformation mechanism further includes a deformation part movable relative to the receiver portion in a travel direction that, when the deformation part is acting on the staple positioned on the receiver portion, extends in a manner that is substantially perpendicular both to a mean plane of the staple and to a longitudinal axis of the bridge of said staple.

17. The instrument according to claim 16, wherein the deformation part further includes a curved distal end having a V-shape extending in the travel direction towards the receiver portion, wherein
while the holder holds the end portions of the bridge of the staple stationary relative to the receiver portion, said distal end exerts on the central portion of said bridge a thrust force causing the central portion to bend into a V-shape.

18. The instrument according to claim 16, wherein the deformation part is assembled to the receiver portion by an assembly that pivots about an axis relative to which the travel direction is substantially circumferential.

19. The instrument according to claim 15, wherein the holder further includes two housings for receiving the end portions of the bridge of the staple, said housings being shaped in the receiver portion to, while the deformation mechanism is acting on the central portion of the bridge, guide the relative movement of respective longitudinal axes of the branches of the staple towards each other while maintaining said axes mutually parallel, and while leaving each branch free to turn about its own axis.
